(19) United States
(12) Patent Application Publication
(10) Pub. No.: US 2021/0022315 A1
(43) Pub. Date: Jan. 28, 2021

(54) METHODS AND COMPOSITIONS WITH RENAL BENEFITS FOR FELINES

(71) Applicant: Société des Produits Nestlé S.A., Vevey (CH)

(72) Inventors: Hsiao-Wei Tu, Platte City, MO (US); George Arthur Bailey Wells, St. Louis, MO (US); Sandra Lyn, ST. LOUIS, MO (US)

(21) Appl. No.: 16/932,288

(22) Filed: Jul. 17, 2020

Related U.S. Application Data
(60) Provisional application No. 62/877,493, filed on Jul. 23, 2019.

(51) Int. Cl. A01K 5/00

(52) U.S. Cl. CPC A01K 5/00

(57) ABSTRACT

A method for treating a feline with renal disease, the method comprising orally administering about 5 to about 70 grams of a pet food composition or pet food compositions to the feline at multiple intervals throughout the day.

### CROSS REFERENCE TO RELATED APPLICATIONS

This application claims priority to U.S. Provisional Application Ser. No. 62/877,493 filed Jul. 23, 2019, the disclosure of which is incorporated in its entirety herein by this reference.

### BACKGROUND

Chronic kidney disease (CKD) is the gradual loss of kidney function over time. CKD is a common disease found in domestic cats and a major cause of morbidity and mortality. The prevalence of CKD increases with advancing age from 5 to 6 years onward and reaches beyond 35% in geriatric cats. Since healthy kidneys are required to filter toxins out of the blood and produce concentrated urine, kidney failure causes waste products and other harmful compounds to accumulate in the blood, resulting in decreased appetite and increased urine volume in cats with CKD.

Because the long-standing damage to the kidneys is irreversible, there is no “cure” for the disease but only an attempt to maintain body weight and slow down the progression of CKD by interventions such as dietary modification, appetite stimulant, and subcutaneous fluid supplement. As a result, most research has been focused on the acceptance and efficacy of prescribed diets or medication. As such, there remains a need for solutions and therapies that can help companion animal with renal diseases such as CKD.

### SUMMARY

The present disclosure relates to methods of treating and providing health benefits to a feline with renal disease. More specifically, the present disclosure relates to providing multiple feedings to a feline having renal disease.

In one embodiment, a method for treating a feline with renal disease can comprise orally administering about 5 to about 70 grams of a pet food composition or pet food compositions to the feline at multiple intervals throughout the day.

Additionally, in another embodiment, a method of increasing daily food intake for a feline having a renal disease can comprise orally administering about 5 to about 70 grams of a pet food composition or pet food compositions to the feline at multiple intervals throughout the day.

Additional features and advantages are described herein and will be apparent from, the following Detailed Description and the Figures.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used in this disclosure and the appended claims, the singular forms “a,” “an” and “the” include plural referents unless the context clearly dictates otherwise. Thus, for example, reference to “an ingredient” or “the ingredient” includes two or more ingredients. The term “and/or” used in the context of “X and/or Y” should be interpreted as “X,” or “Y,” or “X and Y.” Where used herein, the term “example,” particularly when followed by a listing of terms, is merely exemplary and illustrative, and should not be deemed to be exclusive or comprehensive.

As used herein, “about” is understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. A range that is “between” two values includes those two values. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight of the total weight of the composition on a dry matter basis unless expressed otherwise. When reference is made to the pH, values correspond to pH measured at 25° C. with standard equipment.

The terms “food,” “food product” and “food composition” mean a product or composition that is intended for ingestion by an animal and provides at least one nutrient to the animal. The term “pet food” means any food composition intended to be consumed by a companion animal.

The term “companion animal” means a dog or a cat. As used herein, the term “dog” and “canine” can be used interchangeably. As used herein, the term “cat” and “feline” can be used interchangeably.

“Wet food” means a pet food having a moisture content from about 50% to about 90%, and in one aspect, from about 70% to about 90%. “Dry food” means a pet food having a moisture content less than about 20%, and in one aspect, less than about 15%, and in a specific aspect, less than about 10%. “Semi-moist food” means a pet food having a moisture content from about 20% to about 50%, and in one aspect, from about 25% to about 35%. “Kibbles” means pieces of dry or semi-moist pet food which can have a pellet shape or any other shape. Non-limiting examples of kibbles include particulates; pellets; pieces of pet food, dehydrated meat, meat analog, vegetables, and combinations thereof; and pet snacks, such as meat or vegetable jerky, rawhide, and biscuits.

The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. Moreover, the description of some steps as “optional” does not imply that the other steps which are not explicitly described as optional are necessarily required.

Any embodiment disclosed herein can be combined with any other embodiment disclosed herein.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

The term “therapeutically effective amount” means an amount of a compound of the present invention that (i) treats or prevents the particular disease, condition, or disorder, (ii) attenuates, ameliorates, or eliminates one or more symptoms relating thereto of the particular disease, condition, or disorder, or (iii) prevents or delays the onset of one or more symptoms of the particular disease, condition, or disorder described herein. In one embodiment, therapeutically effect amount can refer to the amount to treat any condition relating to a kidney or renal disease.

### EMBODIMENTS

The present disclosure relates to methods of treating and/or providing health benefits to a feline having kidney disease, or otherwise having a condition that reduced food intake. The present inventors have discovered that utilizing multiple feedings for a feline throughout the day provides an unexpected health benefit of increasing food intake as compared to traditional feeding schedules. Such effect can help treat such animals suffering from kidney disease, or other conditions that reduce food intake in the animal. In one embodiment, the feline can have renal disease, cancer, diabetes, hepatitis, anorexia, or a health condition that reduces appetite.

Accordingly, in a general embodiment, a method for treating a feline with renal disease can comprise orally administering about 5 to about 70 grams of a pet food composition or pet food compositions to the feline at multiple intervals throughout the day. Additionally, in another embodiment, a method of increasing daily food intake for a feline can comprise orally administering about 5 to about 70 grams of a pet food composition or pet food compositions to the feline at multiple intervals throughout the day.

Generally, the feline can be fed at multiple intervals throughout the day. In one aspect, the multiple intervals can include at least three separate feedings. In another aspect, the multiple intervals can include at least four separate feedings. In other aspects, the multiple intervals can include at least five, six, or even seven separate feedings throughout the day. As discussed herein, such multiple feedings increases total daily food consumption by the feline as compared to a comparative feeding, wherein the comparative feeding administers the pet food composition at an interval of once per day.

Generally, the composition can be administered on a regular basis to the feline. In one aspect, the regular basis can be at least weekly, or in one specific aspect, at least daily. In one embodiment, the composition can be administered on a long-term basis to the feline. In one aspect, the long-term basis can be at least for 6 months, and in one specific aspect, for at least 1 year, or even for the lifetime of the pet. In another aspect, the administration can be until the animal is cured of the disease. In some embodiments, the composition can be administered to the feline for a time period of at least one week, at least one month, at least two, three, four, five or six months; and in some embodiments, for at least one year, or even for the lifetime of the pet. During the time period, the composition can be administered to the feline at least one day per week, at least two days per week, at least three, four, five or six days per week; or even seven days per week.

Generally, the composition or multiple compositions can be administered in multiple separate intervals per day. In an embodiment, the composition can be administered in each interval in an amount of about 5 to about 70 grams of a pet food. In one aspect, a dry pet food composition can be administered in each interval in an amount of about 5 to about 20 grams of pet food. In another aspect, a wet pet food composition can be administered in each interval in an amount of about 20 to about 40 grams of pet food. In other aspects, a pet food can be administered from about 5, 10, 15, 20, 25, 30, 35, 40, 45, or even 50 grams to about 30, 35, 40, 45, 50, 55, 60, 65, or even 70 grams per interval. In one aspect, the feline can be administered a single pet food throughout the day. In another aspect, the feline can be administered different pet foods throughout the day. Such pet foods can be dry, wet, semi-moist, renal, or any other pet food, including combinations of such foods.

In an embodiment, the pet food compositions can be renal pet foods. In one embodiment, the composition can comprise from about 0.6% to about 0.8% phosphorus on a dry matter basis. In another embodiment, the composition can comprise from about 0.25% to about 0.35% sodium on a dry matter basis. In still another embodiment, the composition can comprise from about 0.75% to about 1% potassium on a dry matter basis. Additionally, the composition can include from 20% to 40% protein, and in some aspects, 20% to 35%, 25% to 30%, 30% to 35%, or even 28% to 30% protein on a dry matter basis. In one aspect, the composition further comprises (i) carnitine, (ii) lysine and methionine, (iii) an antioxidant, such as glutathione, or (iv) mixtures thereof. The composition generally include protein, for example at least about 20 wt %, at least about 25 wt %, or even at least about 30 wt % of the composition. Additionally, the composition can have balanced amounts of magnesium, sodium and potassium; for example, the ratio of potassium to sodium can be about 5:1 to about 1:1, in one aspect, about 5:1 to about 2:1, with the magnesium in an amount of about 0.08 wt % to about 0.25 wt %, and in one aspect, from about 0.10 wt % to about 0.15 wt %. At least a portion of the magnesium, sodium and potassium can be provided as isolated compounds (e.g., salts). Alternatively or additionally, at least a portion of the magnesium, sodium and potassium can be provided by one or more foodstuffs. For example, magnesium can be provided by wheat bran, whole grains, leafy green vegetables, meat, beans and bananas; and potassium and sodium can be provided by meats, fish, whole grains, yogurt, bananas, sweet potatoes, squash, beans and tomatoes.

The pet food compositions disclosed herein can be any food formulated for consumption by a feline. In an embodiment, the pet food composition provides complete nutrition as defined by the Association of American Feed Control Officials (AAFCO) as of Jan. 1, 2019, for a cat. As such, in one aspect, the composition can be a complete and nutritionally balanced pet food. Generally, the pet food composition comprises protein, fat, carbohydrate, and fiber. In various embodiments, the pet food composition can contain 10% to 70% protein, 10% to 70% fat, 5% to 50% carbohydrate, and/or 1% to 30% fiber.

The pet food composition can comprise meat, such as emulsified meat. Examples of suitable meat include poultry, beef, pork, lamb and fish, especially those types of meats suitable for pets. The meat can include any additional parts of an animal including offal. Some or all of the meat can be provided as one or more meat meals, namely meat that has been dried and ground to form substantially uniform-sized particles and as defined by AAFCO. Additionally or alternatively, vegetable protein can be used, such as pea protein, corn protein (e.g., ground corn or corn gluten), wheat protein (e.g., ground wheat or wheat gluten), soy protein (e.g., soybean meal, soy concentrate, or soy isolate), rice protein (e.g., ground rice or rice gluten) and the like.

The pet food compositions disclosed herein can comprise one or more of a vegetable oil, a flavorant, a colorant or water. Non-limiting examples of suitable vegetable oils include soybean oil, corn oil, cottonseed oil, sunflower oil, canola oil, peanut oil, safflower oil and the like. In some embodiments, the lipids in the composition can consist of the MCTs and one or more of any vegetable oil, any fish oil, the lipid from any meat, and any omega-3 fatty acids.

Non-limiting examples of suitable flavorants include yeast, tallow, rendered animal meals (e.g., poultry, beef, lamb, pork), flavor extracts or blends (e.g., grilled beef), animal digests, and the like. Non-limiting examples of suitable colorants include FD&C colors, such as blue no. 1, blue no. 2, green no. 3, red no. 3, red no. 40, yellow no. 5, yellow no. 6, and the like; natural colors, such as caramel coloring, annatto, chlorophyllin, cochineal, betanin, turmeric, saffron, paprika, lycopene, elderberry juice, pandan, butterfly pea and the like; titanium dioxide; and any suitable food colorant known to the skilled artisan.

The pet food compositions disclosed herein can optionally include additional ingredients, such as starches, humectants, oral care ingredients, preservatives, amino acids, fibers, prebiotics, sugars, animal oils, aromas, other oils additionally or alternatively to vegetable oil, salts, vitamins, minerals, probiotic microorganisms, bioactive molecules or combinations thereof.

Non-limiting examples of suitable starches include a grain such as corn, rice, wheat, barley, oats, potatoes, peas, beans, cassava, and the like, and mixtures of these grains, and can be included at least partially in any flour. Non-limiting examples of suitable humectants include salt, sugars, propylene glycol and polyhydric glycols such as glycerin and sorbitol, and the like. Non-limiting examples of suitable oral care ingredients include alfalfa nutrient concentrate containing chlorophyll, sodium bicarbonate, phosphates (e.g., tricalcium phosphate, acid pyrophosphates, tetrasodium pyrophosphate, metaphosphates, and orthophosphates), peppermint, cloves, parsley, ginger and the like. Non-limiting examples of suitable preservatives include potassium sorbate, sorbic acid, sodium methyl para-hydroxybenzoate, calcium propionate, propionic acid, and combinations thereof.

Specific amounts for each additional ingredient in the pet food compositions disclosed herein will depend on a variety of factors such as the ingredient included in the first edible material and any second edible material; the species of animal; the animal's age, body weight, general health, sex, and diet; the animal's consumption rate; the purpose for which the food product is administered to the animal; and the like. Therefore, the components and their amounts may vary widely.

### EXAMPLE

By way of example and not limitation, the following non-limiting study is illustrative of compositions and methods for treating renal disease and increasing food intake in a feline, in one or more embodiments provided by the present disclosure.

### Example 1—Feeding Study for Felines

Twenty-two renal cats were fed three different renal diets, (hereinafter “D1”, “D2”, and “D3”) under the control condition and the test condition. The cats were fed over a period of 6 days. The control method fed the diets once a day and the test method fed the diets three times a days, every 2 hours, with both protocols allowing 6 hours of access to the food per day. The results of food and caloric intake are shown in tables 1 and 2.

TABLE 1

| Diet | Test (kcal) | Control (kcal) | Increase (%) |
|---|---|---|---|
| D1 | 85.19 | 77.83 | 9.46 |
| D2 | 88.38 | 74.42 | 18.76 |
| D3 | 167.56 | 118.92 | 40.90 |
| Total | 341.13 | 271.17 | 25.80 |

TABLE 2

| Diet | Test (grams) | Control (grams) | Increase (%) |
|---|---|---|---|
| D1 | 80.44 | 73.49 | 9.46 |
| D2 | 103.49 | 87.14 | 18.76 |
| D3 | 146.72 | 104.13 | 40.90 |
| Total | 330.65 | 264.76 | 24.89 |

As shown in Tables 1 and 2, cats fed with the test method protocols resulted in more consumed food for each diet compared to tradition feeding methods, as measured both in terms of weight of food and calories consumed.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for treating a feline with renal disease, the method comprising orally administering about 5 to about 70 grams of a pet food composition or pet food compositions to the feline animal at multiple intervals throughout the day.

2. The method of claim 1, wherein the multiple intervals include at least three separate feedings.

3. The method of claim 1, wherein the multiple intervals include at least four separate feedings.

4. The method of claim 1, wherein the administering increases total daily food consumption by the feline as compared to a comparative feeding, wherein the comparative feeding administers the pet food composition or one of the pet food compositions at an interval of once per day.

5. The method of claim 1, wherein the pet food composition is a complete and nutritionally balanced pet food.

6. The method of claim 1, wherein the pet food composition is administered to the feline on a regular basis.

7. The method of claim 1, wherein the administering includes a single pet food.

8. The method of claim 1, wherein the administering includes at least two different pet foods.

9. A method of increasing daily food intake for a feline, the method comprising orally administering about 5 to about 70 grams of a pet food composition or pet food compositions to the feline at multiple intervals throughout the day.

10. The method of claim 9, wherein the multiple intervals include at least three separate feedings per day.

11. The method of claim 9, wherein the method increases total daily food consumption by the feline as compared to a comparative feeding, wherein the comparative feeding administers the pet food composition or one of the pet food compositions at an interval of once per day.

12. The method of claim 9, wherein the pet food composition is a complete and nutritionally balanced pet food and the pet food composition is administered to the feline on a regular basis.

13. The method of claim 9, wherein the feline has renal disease, cancer, diabetes, hepatitis, anorexia, or a health condition that reduces appetite.

14. The method of claim 9, wherein the administering includes a single pet food.

15. The method of claim 9, wherein the administering includes at least two different pet foods.
